(19) United States
(12) Patent Application Publication
(10) Pub. No.: US 2009/0291895 A1
(43) Pub. Date: Nov. 26, 2009

(54) Methods and Compositions for the Treatment of Inflammatory Diseases

(76) Inventors: Scott W. Wong, Beaverton, OR (US); John M Jones, Oceanside, CA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/470,885

(22) Filed: May 22, 2009

Related U.S. Application Data
(60) Provisional application No. 61/055,734, filed on May 23, 2008.

(51) Int. Cl. A61K 38/16

(52) U.S. Cl. 514/12

(57) ABSTRACT

Compositions and methods for treating inflammatory disorders are provided.

This application claims priority under 35 U.S.C. §119(e) to U.S. Provisional Patent Application No. 61/055,734, filed on May 23, 2008. The foregoing application is incorporated by reference herein.

Pursuant to 35 U.S.C. Section 202(c), it is acknowledged that the United States Government has certain rights in the invention described herein, which was made in part with funds from the National Institutes of Health/National Cancer Institute Grant Nos. CA075922 and GHEMA 0109D; National Institutes of Health/U.S. National Institute of Allergy and Infectious Diseases Grant No. DMID-BAA-03-38; and the Department of Defense Grant No. W81XWH-05-1-0046.

### FIELD OF THE INVENTION

The present invention relates to the fields of inflammation. Specifically, compositions and methods for treating and preventing inflammatory disorders are disclosed.

### BACKGROUND OF THE INVENTION

Several publications and patent documents are cited throughout the specification in order to describe the state of the art to which this invention pertains. Each of these citations is incorporated herein by reference as though set forth in full.

Monkeypox virus (MPV) is a member of the genus Orthopoxvirus, which includes variola major, the etiologic agent of smallpox (Shchelkunov et al. (2001) FEBS Lett., 509:66-70; Shchelkunov et al. (2002) Dokl. Biochem. Biophys., 384:143-7; Shchelkunov et al. (2002) Virology, 297:172-94). Monkeypox virus and variola major share considerable homology, approximately 85% at the genomic level, and cause similar disease manifestations in infected humans. Although variola major is no longer a worldwide threat, MPV is as the virus naturally infects rodents and primates in sub-Saharan Africa, and since its discovery, thousands of cases of human MPV infection have been reported. The disease is primarily transmitted from animals to humans, either through animal bites or through direct contact with animal body fluids. Person-to-person transmission is rare (less than ⅓ of reported cases), and is acquired through close contact and exposure to aerosol droplets or contaminated body fluids (Shchelkunov et al. (2001) FEBS Lett., 509:66-70; CDC, Questions and Answers About Monkeypox, 2003). More importantly, MPV infection of humans is clinically indistinguishable from smallpox, sharing similar pathology and disease progression, and without proper medical attention, a 1-10% mortality rate (CDC, Questions and Answers About Monkeypox, 2003). Further complicating diagnosis, the early stages of human MPV infection are often misdiagnosed as chicken pox, caused by varicella-zoster virus. Although smallpox was officially eradicated in 1976 by world-wide vaccination, recent cases of MPV in the United States indicates that MPV should be considered as a reemerging zoonotic infection that poses a threat to the millions of non-vaccinated individuals.

The poxviridae family is characterized as large, DNA viruses that are highly species specific and cause disease in a wide variety of organisms. Many poxviruses encode proteins that inhibit normal chemokine function, collectively, these proteins are referred to as viral chemokine binding proteins (vCBPs) (Alejo et al. (2006) PNAS, 103:5995-6000; Boomker et al. (2005) Cytokine & Growth Factor Rev., 16:91; Holst et al. (2003) Contrib. Microbiol., 10:232-52; Knipe and Howley, eds. Field's Virology. 5th ed., Vol. 2. 2007, Lippincott Williams & Wilkins: Philadelphia). Members of the orthopoxvirus and leporipoxvirus genera express a secreted, 35 kDa protein, commonly referred to as viral CC-chemokine inhibitor (vCCI), vCBP-I, or 35kDa, that binds to human and rodent CC and CXC chemokines with high affinity, competitively inhibiting their normal interaction with cellular chemokine receptors (Smith et al. (1997) Virology, 236:316-27). Members of the myxomavirus genus also encode a secreted CC chemokine inhibitor (referred to as T7 or vCBP-II), additionally, these proteins have also been shown to effectively scavenge γ-IFN (McFadden et al. (2000) Curr. Opin. Microb., 3:371). As a result of their inhibitory nature, all of these secreted proteins function as anti-inflammatory proteins during viral infection. All vCBPs represent a structurally unique family that does not share homology to any known cellular chemokine receptors, or any other mammalian or eukaryotic proteins (Alcami et al. (1998) J. Immunol., 160:624-633; Carfi et al. (1999) PNAS, 96:12379-83; Graham et al. (1997) Virology, 229:12-24; Seet et al. (2001) PNAS, 98:9008-9013). To date, two animal models have been used to investigate the effect vCCI has on poxvirus pathogenesis. Expression of vCCI during experimental vaccinia infection in mice has shown to greatly reduce the number of infiltrating cells in the lungs of vaccinia infected mice (Reading et al. (2003) J. Immunol., 170:1435-42). Additionally, skin lesions from rabbits infected with rabbitpox show reduced infiltrates, compared to a vCCI knockout virus (Graham, et al. (1997) Virology, 229:12-24).

Chemokines belong to a superfamily of small (8-14 kDa) proteins that possess similar structural and functional properties (Murphy, P. M., Chemokines, in Fundamental Immunology, W. E. Paul, Ed. (2003) Lippincott Williams & Wilkins: Philadelphia, 801-840). The chemokine family is further divided into the following subtypes: C, CC, CXC, and CX₃C, based on the position of conserved cysteines located in the N-terminus of the protein. Most of the known chemokines (˜94%) belong to the CXC or CC subtypes. Chemokines impose function by binding to seven transmembrane G-protein-coupled receptors (GPCRs) and glycosaminoglycans (GAGs), initiating downstream signaling events leading to adhesion, contraction, and actin polymerization (Murphy, P. M., Chemokines, in Fundamental Immunology, W. E. Paul, Ed. (2003) Lippincott Williams & Wilkins: Philadelphia, 801-840; Webb et al., (1993) PNAS, 90:7158-62). Although primarily known for their ability to mediate recruitment of effector leukocytes and lymphocytes during injury or pathogenic insult, chemokines are also critically involved in a variety of cellular processes, such as the development of secondary lymphoid tissue, organogenesis, angiogenesis, and hematopoiesis (Murphy, P. M., Chemokines, in Fundamental Immunology, W. E. Paul, Ed. (2003) Lippincott Williams & Wilkins: Philadelphia, 801-840; Rollins, B. J. (1997) Blood, 90:909-28). As a component of both the innate and adaptive immune responses, chemokines have been targeted by viruses who have obtained the ability to modulate and mimic chemokine function.

Along with their role in mediating inflammation due to injury or pathogen, some chemokines can play key roles in the progression of many auto-immune and neurodegenerative diseases, such as rheumatoid arthritis, Grave's disease, multiple sclerosis, Alzheimer's disease, human immunodeficiency virus-associated dementia, Type 1 diabetes, and Parkinson's disease (Gerard et al. (2001) Nat. Immunol., 2:108-15). Most auto-immune diseases involve autoreactive lymphocytes that can express chemokines, such as IL-8, MCP-1, MIP-1α, MIP-1β, and RANTES, which promote the recruitment of inflammatory cells. It is this influx of inflammatory cells and their secreted products which mediate the auto-immune destruction of host cells and tissue, thus promoting disease. Current therapies for treating chemokine-mediated diseases generally involve suppression of the host immune system, but as with any immunosuppressive regime, there is substantial risk for secondary infection. Initially developed as possible blocking agents for HIV infection, small molecule antagonists for chemokine receptors are currently being evaluated in both animal models and clinical trials for effectiveness in treating chemokine-mediated diseases, but to date, no therapies exist that specifically target the chemokine protein itself (Onuffer et al. (2002) Trends Pharmacol. Sci., 23:459-67).

### SUMMARY OF THE INVENTION

In accordance with the present invention, methods for inhibiting inflammation in a patient or treating an inflammatory disorder in a patient are provided. The methods comprise the administration of a composition comprising a monkeypox virus viral CC-chemokine inhibitor (MPV vCCI) and at least one pharmaceutically acceptable carrier. In a particular embodiment, the methods further comprise the administration of at least one additional anti-inflammatory agent.

In accordance with another aspect of the instant invention, compositions for inhibiting inflammation and treating an inflammatory disorder are provided. The compositions comprise a monkeypox virus viral CC-chemokine inhibitor (MPV vCCI), at least one additional anti-inflammatory agent, and at least one pharmaceutically acceptable carrier.

### BRIEF DESCRIPTIONS OF THE DRAWING

FIG. 1 provides an amino acid comparison of MPV vCCI (SEQ ID NO: 7) to vCCIs encoded by variola virus (VARV; SEQ ID NO: 8), cowpox virus (CPV; SEQ ID NO: 9), rabbitpox virus (RPV; SEQ ID NO: 10), and vaccinia Copenhagen strain (VV COP; SEQ ID NO: 11). Alignments were preformed with ClustalW using Blosum scoring matrix. Dark shaded boxes indicate either: 1) identical residues, or 2) unique residues to MPV vCCI. Lightly shaded boxes represent similar residues to MPV vCCI.

FIG. 2A provides images of an immunofluorescence analysis on MPV-infected (left panel) or mock-infected (right panel) BSC₄₀ cells fixed at 24 hours post infection. Cells were stained with a mouse anti-vCCI monoclonal antibody, followed by a biotinylated horse anti-mouse secondary antibody, and visualized using an alexa-488 conjugated to streptavidin. Nuclear staining was performed using Hoescht stain. All images were taken with 20× objective. FIG. 2B provides a Western analysis demonstrating the secretion of MPV vCCI during MPV infection. Samples of supernatants and lysates from MPV (lanes 1 and 3) and Mock (lanes 2 and 4) infected BSC₄₀ cells were resolved on 4-12% Bis-Tris NuPAGEO gels and transferred to PVDF. Western blot analysis was performed using a mouse anti-vCCI monoclonal antibody (3D1) and an HRP-conjugated goat anti-mouse secondary antibody. Purified MPV vCCI was used as a positive control (lane 5).

FIG. 3A provides a stained gel demonstrating that MPV vCCI binds rhesus MIP-1α. Purified MPV vCCI and rhMIP-1α were mixed together at a 1:1 molar ratio and incubated for ten minutes at room temperature. Purified MPV vCCI alone and rhMIP-1α alone were used as controls. Reactions were resolved on a 12% native PAGE gel and stained with SimplyBlue™ Safe Stain. FIG. 3B provides a stained gel wherein MPV vCCI was titrated from limiting to excess, into a reaction mixture with a fixed amount of rhMIP-1α. MPV vCCI alone and rhMIP-1α alone were used as controls. FIG. 3C provides an image a gel which demonstrates the co-immunoprecipitation of rhMIP-1α with MPV vCCI. Increasing amounts of rhMIP-1α (0.1 μg to 2.0 μg—lanes 4 to 8) were incubated with a fixed amount of MPV vCCI (6 μg), as a result more rhMIP-1α co-elutes with immunoprecipitated MPV vCCI. 3 μg of rhMIP-1α (lane 2) and 6 μg of MPV vCCI (lane 1) were used as positive controls. As a negative control, MPV vCCI immunoprecipitation was performed on 3 μg of rhMIP-1α alone (lane 3). Proteins were resolved on 4-12% Bis-Tris NuPAGE® gels.

FIG. 4 is a graph demonstrating the inhibition of rhesus MIP-1α mediated migration of Human THP-1 cells. 5×10⁵ THP-1 cells suspended in 100 μL of assay media (RPMI 1640+0.5% fetal bovine serum) were placed in 3 μm pore size transwell inserts and placed in 24-well culture plates containing 600 μL assay media with 10⁻⁹ M rhMIP-1α plus increasing concentrations of MPV vCCI or 10⁻⁷ M heat inactivated MPV vCCI (ΔMPV vCCI). PBS was used as a negative control. Following a 4 hour incubation at 37° C. (5% CO₂), THP-1 cells migrating through the transwell were counted using a CyQuant® cell proliferation assay kit (Molecular Probes, Eugene, Oreg.). Represented data are the average number of migrated cells of 3 wells (×2500)±SEM.

FIGS. 5A-5D provide images of the in vivo inhibition of rhesus MIP-1α mediated chemotaxis. GELFOAM® sponges containing agarose-embedded (FIG. 5A) rhMIP1α or (FIG. 5B) rhMIP1α+MPV vCCI or (FIG. 5C) PBS were implanted s.c. in the back of a rhesus macaque (≧8 cm apart), where they remained for 7 days before being harvested, sectioned, and stained. CD14 staining shows a clear reduction in CD14⁺ infiltrates in the GELFOAM® sponges containing rhMIP1α+MPV vCCI, as compared to rhMIP1α. An isotopically matched primary antibody was used on a section of rhMIP1α-containing GELFOAM® as an antibody control (FIG. 5D). All images (A-D) were taken using a 20× objective and are the same size (345,000 pixels). FIG. 5E provides a graph of the quantification of CD14⁺ infiltrates (E) performed by comparing the number of DAB+ pixels in each image and normalizing to PBS and represented as a migration index±SEM.

FIG. 6 provides a graph demonstrating that MPV vCCI inhibits relapsing experimental allergic encephalomyelitis (EAE). Following induction of EAE by administration of PLP139-151 peptide±MPV vCCI, mice (n=4) were observed on a daily basis and scored for disease using the following scale: 0—Normal, 0.5—Partially limp tail, 1.0—Paralyzed tail, 2.0—Hind limb paresis, 2.5—One hind limb paralyzed, 3.0—both hind limbs paralyzed, 3.5—Hind limbs paralyzed; fore limbs weak, 4.0—Fore limbs paralyzed, 5.0—Moribund. Represented values are the average scores for all mice within each group. Symbols represent the four groups: ♦—PLP139-151 peptide; ▪—PLP139-151 peptide+MPV vCCI; ▴—MPV vCCI alone control; —buffer alone control.

### DETAILED DESCRIPTION OF THE INVENTION

Infection of rhesus macaques with MPV represents an excellent non-human primate model for variola and for determining how vCCI may contribute to MPV pathogenesis. Herein, the first evidence is provided that MPV vCCI is expressed and secreted during MPV infection and that MPV vCCI interacts with rhesus MIP-1α (rhMIP-1α) in vitro and in vivo inhibiting normal chemokine function. Additionally, the utility of MPV vCCI in treating chemokine-mediated disease is demonstrated as MPV vCCI is shown to inhibit relapsing EAE in mice. This represents a novel therapeutic approach for treating diseases and disorders mediated by chemokine function.

As shown herein, MPV encodes a secreted chemokine binding protein, vCCI, that is abundantly expressed and secreted from MPV infected cells. Electrophoretic mobility shift assay (EMSA) data shows vCCI efficiently binds rhesus MIP-1α (rhMIP-1α) at near one to one stoichiometry. In vitro chemotaxis experiments demonstrate that vCCI completely inhibits rhMIP-1α mediated chemotaxis, while in vivo recruitment assays in rhesus macaques using chemokine-saturated implants show a decrease in the number of CD14⁺ cells responding to rhMIP-1α when vCCI is present, suggesting vCCI is effectively inhibiting chemokine function both in vitro and in vivo. It is also demonstrated herein that vCCI can diminish the severity of the acute phase and completely inhibit the relapsing phase of experimental allergic encephalomyelitis (EAE) disease. These data represent the first in vitro and in vivo characterization of vCCI emphasizing its function as a potent inhibitor of rhMIP-1α. Furthermore, the ability of vCCI to inhibit relapsing EAE disease represents a novel therapeutic approach for treating chemokine-mediated diseases.

### I. Definitions

The following definitions are provided to facilitate an understanding of the present invention:

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% or more by weight of the given compound. Purity is measured by methods appropriate for the given compound (e.g. chromatographic methods, agarose or polyacrylamide gel electrophoresis, HPLC analysis, and the like).

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

As used herein, the terms “inflammatory disease” and “inflammatory disorder” (which are used interchangeably herein) refer to a disease or disorder caused by or resulting from or resulting in inflammation. The term “inflammatory disease” may also refer to a dysregulated inflammatory reaction that causes an exaggerated response by macrophages, granulocytes, and/or T-lymphocytes leading to abnormal tissue damage and cell death. Preferably, the inflammatory disease or disorder is associated with chemokine-medicated trafficking of leukocytes or other inflammatory cells. Essentially, any disease or disorder which is etiologically linked to the pro-inflammatory process and cellular infiltration due to chemokines (e.g., CC-chemokines, particularly MIP-1α) would be considered susceptible to treatment. An “inflammatory disease” can be either an acute or chronic inflammatory condition and can result from infections or non-infectious causes. Inflammatory diseases and disorders include, without limitation, inflammatory lesions (e.g., those associated with multiple sclerosis, graft or organ transplant rejection, tuberculosis, and the like), atherosclerosis, arteriosclerosis, autoimmune disorders, erythematosis, multiple sclerosis, systemic lupus erythematosus, polymyalgia rheumatica (PMR), gouty arthritis, degenerative arthritis, tendonitis, bursitis, psoriasis, eczema, dermatitis, cystic fibrosis, arthrosteitis, rheumatoid arthritis, inflammatory arthritis, Sjogren's Syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroditis, Graves' disease, Goodpasture's disease, mixed connective tissue disease, sclerosing cholangitis, inflammatory bowel disease, colitis, Crohn's Disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, emphysema, hayfever, allergies, acute anaphylaxis, rheumatic fever, glomerulonephritis, pyelonephritis, cellulitis, cystitis, chronic cholecystitis, ischemia (ischemic injury and ischemia-reperfusion), hypertension, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, and vulvovaginitis, angitis, chronic bronchitis, osteomylitis, optic neuritis, temporal arteritis, transverse myelitis, necrotizing fascilitis, necrotizing enterocolitis, infection-related disorders such as acute and chronic bacterial and viral infections and sepsis, and neoplasia (including leukocyte recruitment in cancer and angiogenesis). In a particular embodiment, the inflammatory disorder is the inflammatory lesions associated with multiple sclerosis, organ transplant rejection, or tuberculosis.

As used herein, an “anti-inflammatory agent” refers to compounds for the treatment of an inflammatory disease or the symptoms associated therewith. Anti-inflammatory agents include, without limitation, vCCIs other than monkeypox vCCI (e.g., from vaccinia), non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin (see, e.g., Migita et al., Clin. Exp. Immunol. (1997) 108:199-203; Migita et al., Clin. Exp. Immunol. (1996) 104:86-91; Foroncewicz et al., Transpl. Int. (2005) 18:366-368), high density lipoproteins (HDL) and HDL-cholesterol elevating compounds (see, e.g., Birjmohun et al. (2007) Arterioscler. Thromb. Vasc. Biol., 27:1153-1158; Nieland et al. (2007) J. Lipid Res., 48:1832-1845; Bloedon et al. (2008) J. Lipid Res., Samaha et al. (2006) Arterioscler. Thromb. Vasc. Biol., 26:1413-1414, which discloses the use of rosiglitazone as an anti-inflammatory, Duffy et al. (2005) Curr. Opin. Cardiol., 20:301-306), rho-kinase inhibitors (see, e.g., Hu, E. (2006) Rec. Patents Cardiovasc. Drug Discov., 1:249-263), anti-malarial agents (e.g., hydroxychloroquine), acetaminophen, glucocorticoids, steroids, beta-agonists, anticholinergic agents, methyl xanthines, gold injections, sulphasalazine, penicillamine, anti-angiogenic agents, dapsone, psoralens, anti-viral agents, statins (see, e.g., Paraskevas et al. (2007) Curr. Pharm. Des., 13:3622-36; Paraskevas, K. I. (2008) Clin. Rheumatol. 27:281-287), and antibiotics. In a particular embodiment, the anti-inflammatory agent is an NSAID or a steroid (e.g., glucocorticoid or corticosteroid).

A “therapeutically effective amount” of a compound or a pharmaceutical composition refers to an amount effective to prevent, inhibit, treat, or lessen the symptoms of a particular disorder or disease. The treatment of an inflammatory disorder herein may refer to curing, relieving, and/or preventing the inflammatory disorder, the symptom of it, or the predisposition towards it.

### II. Methods of Treatment

The present invention encompasses compositions comprising MPV vCCI and at least one pharmaceutically acceptable carrier. The composition may further comprise at least one other anti-inflammatory agent. Such composition may be administered, in a therapeutically effective amount, to a patient in need thereof for the treatment of an inflammatory disease or disorder. In a particular embodiment, at least one other anti-inflammatory agent is administered separately from the above composition (e.g., sequentially or concurrently).

The compositions of the present invention can be administered by any suitable route, for example, by injection (e.g., for local (direct) or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intraocular, intrapulmonary, intraareterial, intrarectal, intramuscular, and intranasal administration. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween 80, Polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435 1712 which are herein incorporated by reference. The pharmaceutical composition of the present invention can be prepared, for example, in liquid form, or can be in dried powder form (e.g., lyophilized).

In a particular embodiment, the composition may be administered topically. The composition for topical administration may be formulated, for example, as a cream, lotion, foam, or ointment. As another example, inflammations of the joints or tendons (e.g., arthritis, tendonitis) may be treated by injecting the composition directly into the affected location. Such injections may be administered at intervals until inflammation has subsided. As yet another example, inflammatory conditions of the airways or lungs (e.g., asthma) may be treated by inhalation therapy with an aerosol formulation of the composition. As still another example, inflammations or autoimmune diseases of the gastrointestinal tract (e.g., irritable bowel syndrome, Crohn's Disease) may be treated orally with the composition formulated as a pill, powder, capsule, tablet, or liquid to coat the lumenal surface of the gastrointestinal tract. As another example, the composition may be administered systemically (e.g., intravenously) for treatment of inflammatory disorders that are, at least in part, systemic in nature (e.g., systemic lupus, multiple sclerosis, rheumatoid arthritis, dermatomyositis and scleroderma) or that do not lend themselves well to localized drug delivery.

In yet another embodiment, the pharmaceutical compositions of the present invention can be delivered in a controlled release system, such as using an intravenous infusion, an implantable osmotic pump, a transdermal patch, liposomes, or other modes of administration. In a particular embodiment, a pump may be used (see Langer, supra; Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61; see also Levy et al., Science (1985) 228:190; During et al., Ann. Neurol. (1989) 25:351; Howard et al., J. Neurosurg. (1989) 71:105). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115 138). In particular, a controlled release device can be introduced into an animal in proximity to the site of inappropriate inflammation. Other controlled release systems are discussed in the review by Langer (Science (1990) 249:1527 1533).

The composition of the instant invention may be administered for immediate relief of acute symptoms or may be administered regularly over a time course to treat the inflammatory disorder. The dosage ranges for the administration of the MPV vCCI of the invention are those large enough to produce the desired effect (e.g., curing, relieving, and/or preventing the inflammatory disorder, the symptom of it, or the predisposition towards it). The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications. In a particular embodiment, the dosage can vary from about 10 μg to about 100 μg per dosage, wherein the dosage can be administered at least once per day and for at least one day.

In yet another embodiment, the MPV vCCI may be modified with polymers such as polyethylene glycol. Such modifications may be used to extend serum half-life or reduce antigenicity of vCCI (see, e.g., U.S. Pat. Nos. 7,022,673 and 7,329,516; Lee et al. (1999) Bioconjugate Chem., 10:973-981; Clark et al. (1996) J. Biol. Chem., 271:21969-21977).

MPV vCCI proteins of the present invention may be prepared in a variety of ways, according to known methods. The proteins may be purified from appropriate sources, e.g., infected hosts, tissues and/or cells, transformed bacterial or animal cultured cells or tissues. The availability of nucleic acid molecules encoding MPV vCCI protein enables production of the protein using in vitro expression methods and cell-free expression systems known in the art. In vitro transcription and translation systems are commercially available. Alternatively, larger quantities of MPV vCCI protein may be produced by expression in a suitable prokaryotic or eukaryotic system. For example, part or all of a DNA molecule encoding for MPV vCCI may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences. MPV vCCI protein produced by gene expression in a recombinant prokaryotic or eukaryotic system may be purified according to methods known in the art. A commercially available expression/secretion system can be used, whereby the recombinant protein is expressed and thereafter secreted from the host cell, and readily purified from the surrounding medium. Affinity separation may be used to isolate and purify MPV vCCI, such as by immunological interaction with antibodies that bind specifically to the recombinant protein or other agents which bind an affinity tag added to the recombinant protein (e.g., nickel columns for isolation of recombinant proteins tagged with 6-8 histidine residues at their N-terminus or C-terminus; alternative tags include the FLAG epitope, the hemagglutinin epitope, and the like). Such methods are commonly used by skilled practitioners.

MPV vCCI protein of the invention, prepared by the aforementioned methods, may be analyzed according to standard procedures. For example, such protein may be subjected to amino acid sequence analysis, according to known methods.

An exemplary amino acid sequence of MPV vCCI is SEQ ID NO: 7. A MPV vCCI amino acid sequence may have at least 90%, 95%, 97%, or 99% homology with SEQ ID NO: 7.

The instant method also encompasses methods of inhibiting MIP-1α, in vitro and/or in vivo. The methods comprise contacting MIP-1α with MPV vCCI.

The following example provides illustrative methods of practicing the instant invention, and is not intended to limit the scope of the invention in any way.

### EXAMPLE

### Materials and Methods

### Protein Alignments

Protein alignments were performed using ClustalW from MacVector version 9.0 software (Accelrys, Inc., Madison, Wis.). A Blosum scoring matrix was used in pairwise alignment of each sequence, with a gap introduction penalty of 10 and a gap extension of 0.1.

Virus, Cell Culture, and MPV vCCI Specific Antibodies

Human monkeypox virus (MPX V79-I-005) was obtained from the Center for Disease Control and Prevention (Atlanta, Ga.) and propagated in BSC₄₀ cells (African green monkey kidney cells—American Type Culture Collection (ATCC), Manassas, Va.) cultured in Dulbecco's modified Eagle's medium (DMEM, Mediatech, Herndon, Va.) supplemented with 10% heat-inactivated fetal bovine serum (HyClone, Logan, Utah), 1% penicillin, streptomycin, and L-glutamine (Invitogen, Carlsbad, Calif.). Viral titers were determined by plaque assay. HeLa cells and primary rhesus fibroblasts were maintained in DMEM and human THP-1 cells were maintained in RPMI 1640 (Mediatech, Herndon, Va.), both supplemented with 10% heat-inactivated fetal bovine serum, 1% penicillin, streptomycin, L-glutamine. RPMI 1640 was further supplemented with HEPES, and sodium pyruvate, 2% sodium bicarbonate (Invitogen, Carlsbad, Calif.). MPV vCCI specific monoclonal antibodies were made onsite in the monoclonal antibody core at the Vaccine and Gene Therapy Institute (Beaverton, Oreg.) using purified recombinant MPV vCCI (see below) as antigen.

### Immunofluorescence Analysis

Approximately 0.8×10⁵ BSC₄₀ cells were seeded onto 12 mm glass cover slips (Fisher Scientific, Pittsburgh, Pa.). The following day, cells were either infected with MPV at a multiplicity of infection (MOI)=1 or mock and at 24 hours post-infection, cells were fixed with 4% paraformaldehyde in PBS at 25° C. for 20 minutes. Fixed cells were then permeabilized with 0.2% triton-x 100 in PBS. Staining for MPV vCCI was performed using mouse monoclonal antibodies (Clone #11A3.4.2), followed by a biotinylated horse anti-mouse secondary antibody (Dako, Cuppertino, Calif.). The 11A3.4.2 clone was used specifically for immunofluorescence because of its low background in this application. Visualization was performed using streptavidin conjugated to Alexa-488 (Invitrogen, Carlsbad, Calif.) followed by a nuclear counterstain with a Hoechst dye (Sigma, St. Louis, Mo.).

### Immunoprecipitation and Western Blot Analysis

2.5×10⁶ BSC₄₀ cells were infected with MPV at MOI=10. Following 24 hours of incubation, supernatants were clarified and concentrated 10-fold via 5,000 MWCO Amicon® Ultra centrifugal filtration device (Millipore, Bedford, Mass.), while infected cells were washed with PBS and lysed in ice cold RIPA buffer (PBS, 1% NP40, 1% sodium deoxycholate, 0.1% sodium dodecyl sulfate). An MPV vCCI-specific mouse monoclonal antibody (clone #3D1) was added to the concentrated supernatants at 12.5 μg/mL and incubated for 1 hour at 4° C. with agitation. 100 μL Protein A/G-plus agarose (Santa Cruz Biotechnology Inc., Santa Cruz, Calif.) was added to the mixture and allowed to incubate for 1 hour at 4° C. with agitation. Protein bound agarose was pelleted and washed twice with cold PBS. Bound proteins were denatured by adding 2× NuPAGE® LDS sample buffer (Invitrogen, Carlsbad, Calif.) and heating to 70° C. for 10 minutes. Proteins (15 μL load) were resolved on 4-12% NuPAGE® Bis-Tris polyacrylamide gels and wet transferred to PVDF membranes at 30V for 1 hour. Protein blots were probed using an anti-MPV vCCI mouse monoclonal antibody (clone #3D1) followed by a horseradish peroxidase-conjugated goat anti-mouse secondary antibody (1:2,000) (Santa Cruz Biotechnology Inc., Santa Cruz, Calif.). Bands were visualized using chemiluminescence. For the co-immunoprecipitation of rhMIP-1α with MPV vCCI, 6 μg of recombinant MPV vCCI was mixed with increasing amounts of recombinant rhMIP-1α (from 0.1 μg to 2 μg). Following 10 minute room temperature incubation, 10 μg of anti-MPV vCCI mouse monoclonal antibody (clone #3D1) was added to the reaction and immunoprecipitation was carried out as described above. Western blot analysis for rhMIP-1α was conducted in a similar fashion as described for MPV vCCI using a cross-reactive human MIP-1α polyclonal antibody (# BAF270—R & D Systems, Minneapolis, Minn.)

Cloning and Expression of Recombinant MPV vCCI

The coding sequence for MPV-J1L was isolated from MPV genomic DNA via PCR using primers specific for MPV-J1L which also contained a 6×-histidine tag (underlined region) and restriction sites for NdeI (5′-CATATGATCCCTACCAGTCTTCAGCA-3′; SEQ ID NO: 1) and XhoI (5′-CTCGAGTCATCAGTGGTGGTGGTGGTGGTGGACACATGCTTTGAGTTTTGT-3′; SEQ ID NO: 2). A non-sense mutation (in quotation marks) was introduced into an internal NdeI site via site directed mutagenesis using the following primers: 5′-AACAAACATCA″C″ATGGGAATCG-3′ (SEQ ID NO: 3) and 5′-CGATTCCCAT″G″TGATGTTTGTT-3′ (SEQ ID NO: 4). A 6×-histidine tagged rhMIP1α was isolated in a similar manner from another expression plasmid also using NdeI (5′-CATATGGCTGACACCCCGACCTC-3′; SEQ ID NO: 5) and XhoI (5′-CTCGAGTCATCAGTGGTGGTGGTGGTGGTGCACGGCACTCAGCTCTAGGTC-3′; SEQ ID NO: 6). The resulting products were cloned into pRSETb (Invitrogen, Carlsbad, Calif.) for expression. Rosetta 2® DE3 cells (Novagen, Madison, Wis.) were transformed with the pRSETb expression plasmids. Expression cultures were set up by diluting overnight cultures 1:20 into 1 L of LB media without antibiotic and incubated for 3 hours at 37° C. with agitation. At 3 hours, the temperature of the cultures was reduced to 25° C. and protein expression was induced with 0.5 μM isopropyl-β-D-thiogalactoside (IPTG; Fisher, Fair Lawn, N.J.) with continued agitation for 6 hours. Cells pellets were harvested by centrifugation (5,000×g for 12 minutes) and stored at −80° C. until use.

Purification of Recombinant MPV vCCI

Induced cell pellets were resuspended in lysis buffer (300 mM NaCl, 50 mM NaPO₄, 20 mM Tris-HCl, 0.1 mM PMSF, 3 mM βME, pH 8.0) and lysed by 2 freeze/thaw cycles, incubation with 1 mg/ml lysozyme, 5 μg/ml DNAase, and 5 μg/ml RNAase for 30 minutes on ice, and then sonicated 30 seconds (3×). Lysates were separated into soluble and insoluble fractions by centrifugation at 20,000×g for 60 minutes at 4° C. Proteins were purified via immobilized metal affinity chromatography (IMAC) by applying the soluble fraction to pre-equilibrated BD Talon® metal affinity resin (Clontech Laboratories Inc, Mountain View, Calif.) (1 ml resin per 2 L culture), where it was incubated on a rotator at room temperature for 1 hour. Protein-bound resin was pelleted and washed (2×) with 20 ml wash buffer (300 mM NaCl, 50 mM NaPO₄, 20 mM Tris-HCl, 10% glycerol, 3 mM βME, pH 7.5). Protein was eluted from the resin by adding 3 ml elution buffer (300 mM NaCl, 50 mM NaPO₄, 20 mM Tris-HCl, 250 mM imidazole, 3 mM βME, pH 7.0) and incubated on a rotator at room temperature for 5 minutes (3×). Eluted protein was 0.22 μm filtered and run over a HiPrep 16/60 Sephacryl S-100 HR column (GE Healthcare, Piscataway, N.J.) pre-equilibrated in running buffer (20 mM NaPO₄, 150 mM NaCl, 3 mM βME, pH 7.0). Pooled fractions were further purified and concentrated by binding to a HiTrap Q FF column and eluted with a 0-1 M NaCl gradient over 20 ml. Protein purity and size were determined on 4-12% Bis-Tris NuPAGEO gels and the purest fractions were pooled together. Endotoxin levels were assessed using a limulus amebocyte lysate (LAL) assay (Cambrex, Walkersville, Md.), followed by endotoxin removal using AffintyPak™ Detoxi-Gel™ endotoxin removal gel (Pierce, Rockford, Ill.). Protein concentration was determined by absorbance spectroscopy. Purified proteins were lyophilized and stored at −80° C., while reconstituted protein was kept at −20° C.

### Electrophoretic Mobility Shift Assays

Purified recombinant rhMIP-1α and MPV vCCI were incubated together at room temperature for 10 minutes. Samples were resolved on a non-denaturing, non-reducing 12% polyacrylamide gel at 30 mA. Bands were visualized using SimplyBlue® SafeStain (Invitrogen, Carlsbad, Calif.).

### In Vitro Chemotaxis Inhibition Assay

Inhibition of THP-1 cell migration was carried out using Transwell® plates (6.5 mm×3.0 μm pore, Corning, New York, N.Y.) equilibrated in assay media (RPMI 1640 supplemented with 0.5% heat-inactivated fetal bovine serum) for 1 hour prior to assay. Ten minutes prior to beginning the assay, 10⁻⁹ M rhMIP-1α was mixed with increasing amounts of MPV vCCI and incubated at 25° C. The protein mixture was then added to 600 μL of assay media in the lower chamber. 5×10⁵ THP-1 cells suspended in 100 μl were added to the upper chamber of the transwell and incubated for 4 hours at 37° C. with 5% CO₂. Migrated cells were counted using CyQuant cell proliferation assay kit (Invitrogen, Carlsbad, Calif.).

### In Vivo Chemotaxis Assay

In vivo recruitment assay was adapted from a previously published angiogenesis assay (Fan et al. (2004) Cancer Res., 64:3186-90). GELFOAM® plugs (Pharmacia & Upjohn Company, Kalamazoo, Mich.) were cut 5 mm³ and rehydrated overnight in PBS at 4° C. On the day of implantation, plugs were briefly dried between two pieces of filter paper and soaked with a) 500 ng of rhMIP-1α, b) 500 ng of rhMIP-1α plus 1.5 μg MPV vCCI (1:1 molar ratio), or c) PBS mixed with 0.4% agarose warmed to 42° C. The soaked implants were stored at 4° C. until implantation. For implantation, rhesus macaques are anesthetized with ketamine (15-20 mg/kg i.m.), placed in oblique ventral recumbency, and the hair clipped from the midscapular region to the shoulder. Skin prep was performed in routine fashion with betadine scrub and solution, followed by placement of a medium fenestrated drape. A 5-10 mm skin incision was made in the lateral mid-scapular region, the skin is undermined with a Kelly forceps for a distance of approximately 2-3 cm from the incision, and the GELFOAM® implants were inserted in the undermined space. The skin was then closed with several simple interrupted sutures. Spacing between implants was maximized to avoid potential functional overlap. The implants remained in the animal for 7 days, at which time, the GELFOAM® plugs and surrounding tissue were excised and cryopreserved in tissue freezing media (Triangle Biomedical Sciences, Durham, N.C.) and stored at −80° C. for later sectioning. All aspects of the experimental implantation studies were performed according to institutional guidelines for animal care and use at the Oregon Health & Science University, West Campus.

### Immunohistochemistry

10 μm sections of the cryopreserved samples were cut and mounted onto Superfrost®/Plus slides (Fisher Scientific, Pittsburgh, Pa.) at room temperature overnight. Slides were fixed with ice cold acetone for 10 minutes and then washed three times with tris-buffered saline (pH 7.4)+0.1% tween-20 (TBST) to remove freezing media. Slides were blocked with PBS+1% BSA and 10% donkey serum at room temperature for 1 hour, followed by PBS+0.3% H₂O₂. A CD14-specific mouse monoclonal primary antibody (Clone # M5E2—BD Pharmingen, San Diego, Calif.) diluted in PBS+1% BSA was incubated on the sections overnight at room temperature. Following TBST washes, sections were incubated with horse anti-mouse secondary antibody conjugated to horse radish peroxidase for 1 hour at room temperature. CD14 specific staining was visualized using a DAB substrate kit (Dako, Cuppertino, Calif.) and counterstained with Hematoxylin QS (Vector Laboratories, Burlingame, Calif.).

### Experimental Allergic Encephalomyelitis (EAE) Model

The EAE model used herein strictly follows the published protocol of Stromnes and Goverman (Nat. Protoc. (2006) 1:1810-9) and was performed according to institutional guidelines for animal care and use at the OHSU, West Campus. Briefly, on day zero, 8 week old, female SJL/J mice (Jackson Labs, Bar Harbor, Mass.) were injected subcutaneously (s.c.) with 200 μg of myelin proteolipid peptide residues 139-151 (PLP139-151); Peptides Intl., Louisville, Ky.) emulsified in complete Freud's adjuvant (Sigma, St. Louis, Mo.), and 100 ng of pertussis toxin (List Biological Laboratories, Inc., Campbell, Calif.) was given intraperitoneally (i.p.), these mice serve as positive controls. Each mouse in the experimental group received an additional 25 μg of MPV vCCI i.p. Mice receiving 25 μg MPV vCCI alone or buffer alone serve as negative controls. On day 3, an additional boost of 100 ng of pertussis and 25 μg of MPV vCCI were given to the appropriate groups. Mice were monitored daily and disease was scored using the following scale: 0—Normal, 0.5—Partially limp tail, 1.0—Paralyzed tail, 2.0—Hind limb paresis, 2.5—One hind limb paralyzed, 3.0—both hind limbs paralyzed, 3.5—Hind limbs paralyzed; fore limbs weak, 4.0—Fore limbs paralyzed, 5.0—Moribund. Additional care was given to mice exhibiting disease, such as, soaked chow and the administration of s.c. fluids to mice exhibiting a 25% reduction in weight.

### Results

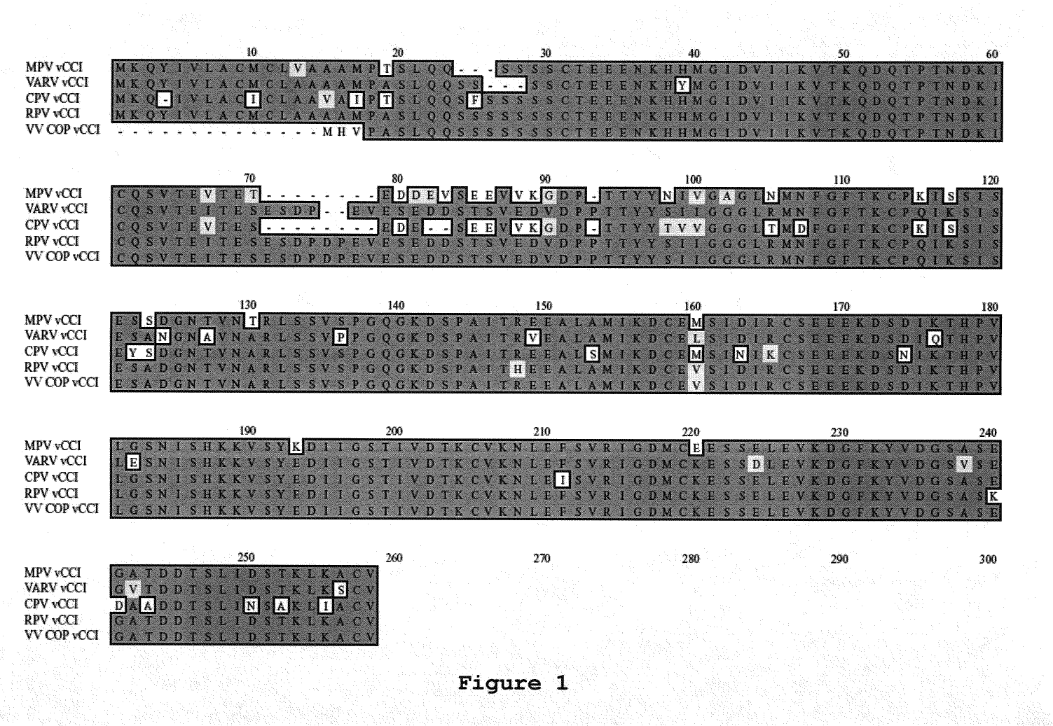

The predicted product of the MPV ORF-J1L is a 27.6 kDa protein, MPV vCCI. The amino acid sequence of MPV vCCI was aligned with other vCCI sequences encoded by variola virus (VARV), cowpox virus (CPV), rabbitpox virus (RPV), and vaccinia stain Copenhagen (VV COP) to determine the level of amino acid sequence homology. FIG. 1 shows the protein alignments for all five proteins and confirms conserved homology between them. On average, MPV vCCI shares approximately 85.8% similarity and 82.5% identity with the other chemokine inhibitors (Table 1). Although highly homologous, there is one area of divergence from amino acid 72 to 94, where the vCCIs of MPV and CPV differ from the other viral vCCIs.

TABLE 1
Homology of MPV vCCI to CPV vCCI, RPV vCCI,
VARV vCCI, and VV COP vCCI.

| vCCI | % Identical to MPV vCCI | % Similar to MPV vCCI |
|---|---|---|
| CPV | 79 | 84 |
| RPV | 85 | 89 |
| VARV | 83 | 87 |
| VV COP | 83 | 83 |

The DNA sequence encoding MPV vCCI was amplified by PCR and a 6× histidine tag was placed in frame at the C-terminus for purification purposes. After a multi-step purification protocol, SDS-PAGE on fractions from anion exchange chromatography shows purified recombinant MPV vCCI. Despite having a predicted molecular weight of 27.6 kDa, MPV vCCI migrates roughly 5-6 kDa higher on SDS-PAGE, which is consistent with other vCCI species, like VARV, CPV, and VV COP, and is more than likely the result of charged residues in the primary sequence.

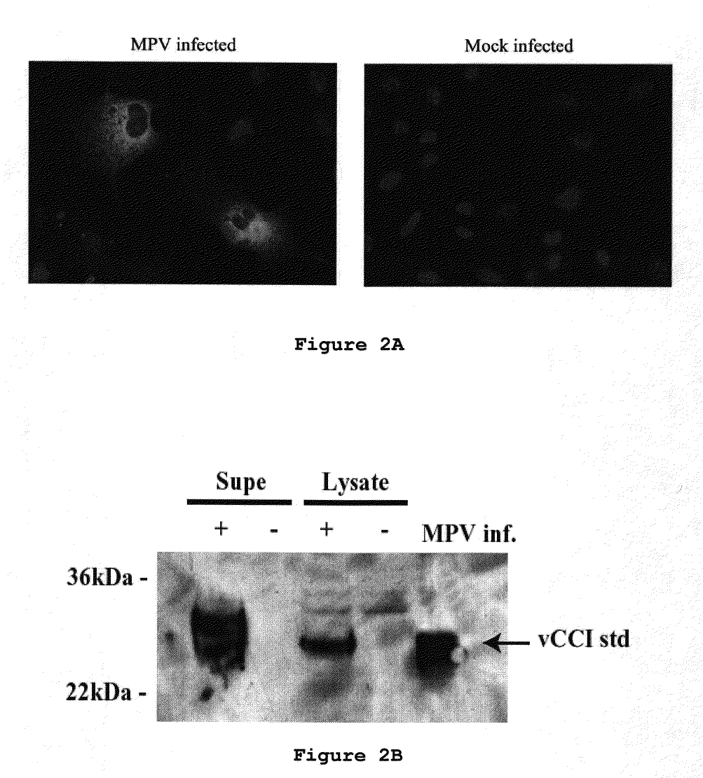

To determine if MPV vCCI protein is expressed during MPV infection, an immunofluorescence assay was performed on MPV infected BSC₄₀ cells using an MPV vCCI specific mouse monoclonal antibody (11A3.4.2). As shown in FIG. 2A, MPV infected cells begin to stain positive for MPV vCCI, as early as 24 hours post infection. Positive cells show an intense cytoplasmic staining as compared to mock infected cells. Next, to determine if MPV vCCI is secreted from MPV infected cells, western blot analysis was performed on clarified/concentrated supernatants and cellular lysates from BSC₄₀ cells infected with MPV for 24 hours. Western blot analysis shows the presence of a MPV vCCI specific band at, or near the apparent molecular weight of ˜35 kDa in supernatant from infected samples, but not in supernatants from mock samples (FIG. 2B). Recombinant MPV vCCI was loaded as a positive control. Taken together, these data clearly demonstrate MPV vCCI is expressed and secreted during MPV infection, either via active transport or during cell lysis.

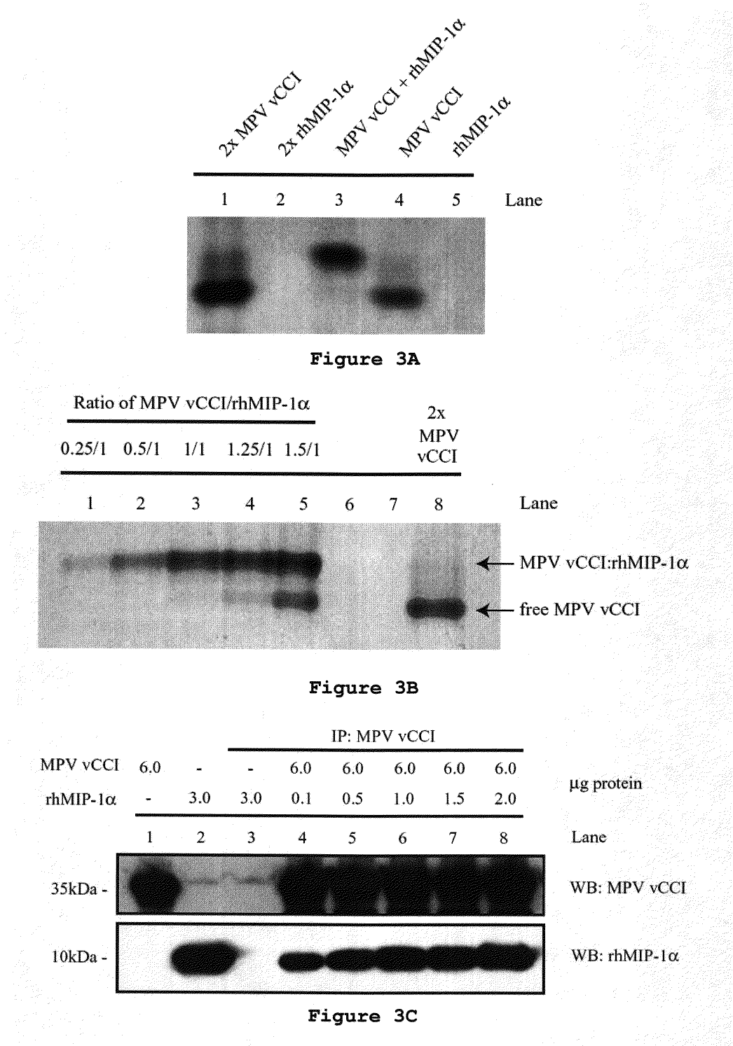

To assess the ability of MPV vCCI to bind rhMIP-1α, a modified electrophoretic mobility shift assays (EMSA) was utilized to visualize differences in MPV vCCI mobility with and without rhMIP-1α present (FIG. 3A). Because of its small size and amino acid content, rhMIP-1α does not stain at the concentrations used (lanes 2 and 5). Therefore, if MPV vCCI is forming a complex with rhMIP-1α, an increase in the apparent molecular weight (MWₐₚₚ) of MPV vCCI should be observed. Compared to free MPV vCCI (lane 4), MPV vCCI runs at a higher MWₐₚₚ when incubated with rhMIP-1α (lane 3). Moreover, to address MPV vCCI aggregation as a possible explanation for the shift in molecular weight, twice the amount MPV vCCI was loaded (lane 1), and although some “smearing” is observed, the higher molecular weight band is not observed. To confirm the presence of both MPV vCCI and rhMIP-1α, the shifted band (lane 3) was excised and in-gel trypsin digest was performed, followed by mass spectrophotometry. Following analysis of unique peptide hits, the presence of two species, MPV vCCI and rhMIP-1α, was confirmed.

To further demonstrate the formation of the MPV vCCI: rhMIP-1α complex, a titration assay was set-up where increasing amounts of MPV vCCI were incubated against a fixed amount of rhMIP-1α. FIG. 3B shows that with limiting amounts of MPV vCCI, the only species present is the higher MWₐₚₚ species (lanes 1 and 2). As MPV vCCI begins to be in excess, the presence of the free MPV vCCI begins to be seen (lanes 4 and 5). As seen in FIG. 3A, 2×MPV vCCI was loaded to verify that aggregation was not the reason for the shifted band (lane 8).

In order to confer specificity, a co-immunoprecipitation assay was performed on a mixture MPV vCCI and rhMIP-1α using an anti-MPV vCCI monoclonal. As shown in FIG. 3C, as increasing amounts of rhMIP-1α were added to the incubation mixture, more rhMIP-1α is co-immunoprecipitated with MPV vCCI (lanes 4-8). This effect is dependent on MPV vCCI, as rhMIP-1α alone does not immunoprecipitated with the MPV vCCI antibody (lane 3). Taken together, these data show that MPV vCCI binds and forms a complex with rhMIP-1α.

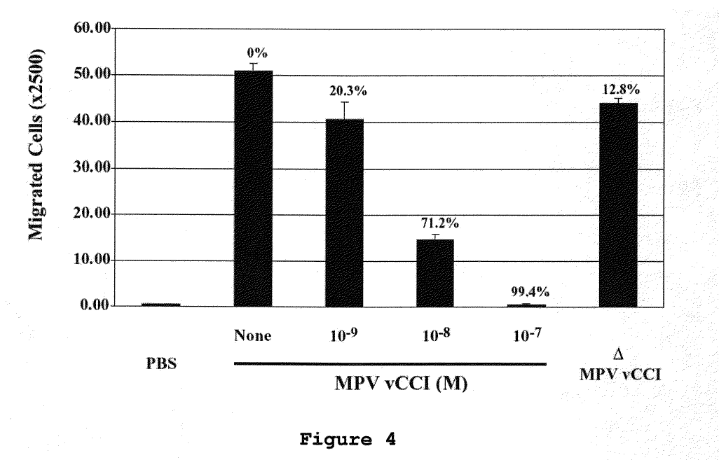

In order to assess the inhibitory properties of MPV vCCI, an in vitro transwell assay using human THP-1 cells, a premonocytic cell line, was utilized. THP-1 cells were used for their consistency, as opposed to isolating cells from different rhesus macaques and dealing with animal to animal variability. Furthermore, is has been previously determined that THP-1 cells are fully responsive to rhMIP-1α with maximum chemotaxis occurring at 10⁻⁹ M. FIG. 4 shows that with increasing concentrations of MPV vCCI, rhMIP-1α mediated chemotaxis is reduced to levels similar to PBS controls. The use of heat inactivated MPV vCCI restores rhMIP-1α mediated migration confirming that the observed effect is mediated by MPV vCCI. These findings clearly show that MPV vCCI is binding to rhMIP-1α and effectively inhibiting chemotaxis.

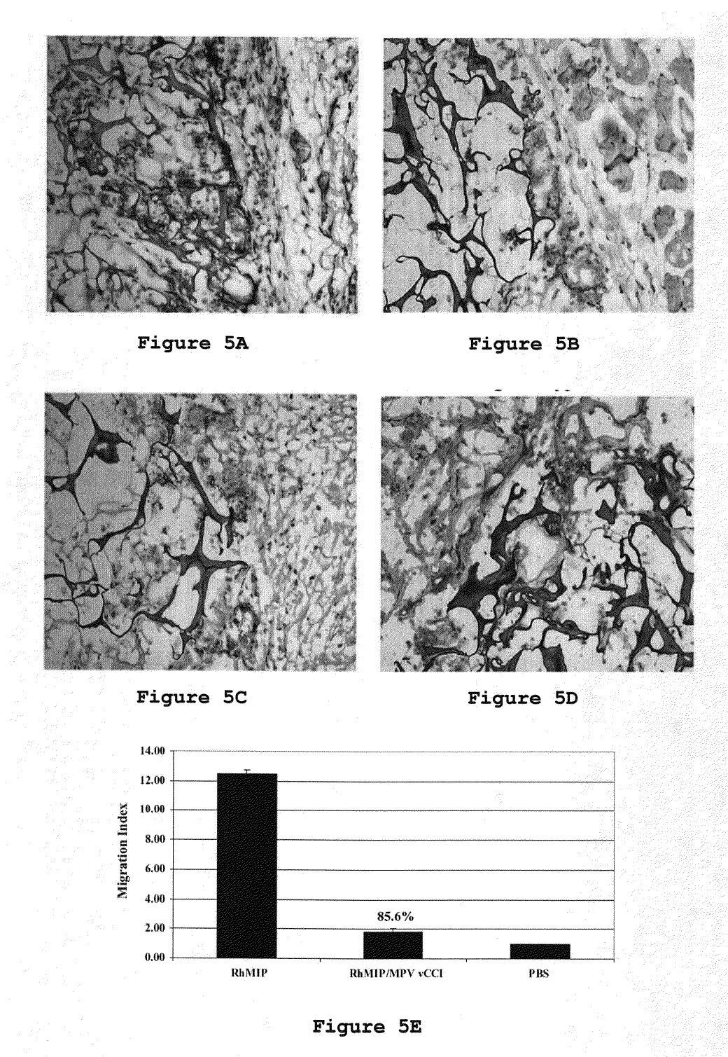

To better understand the in vivo function of MPV vCCI, an in vivo assay was designed to observe whether or not MPV vCCI could effectively inhibit rhMIP-1α mediated recruitment. To introduce samples into a macaque in a controlled setting, a previously published angiogenesis protocol by Fan et al. (Fan et al. (2004) Cancer Res., 64:3186-90) was modified. GELFOAM® is an inert, sponge-like material used as a hemastatic material during surgery. When a soluble agent, such as a chemokine, is absorbed into GELFOAM® in the presence of 0.4% agarose, it can be handled as a solid and once implanted is slowly released into the external environment over time. Based on previous work that showed rhMIP-1α mediates recruitment of CD14+ cells during in vivo recruitment assays, rhMIP-1α was incubated with MPV vCCI at a 1:1 molar ratio prior to absorption into GELFOAM® plugs. Following surgical implantation, incubation, and subsequent removal of the protein-saturated implants, frozen sections of the GELFOAM® implants and surrounding tissue were analyzed by immunohistochemistry using a CD14 specific antibody. In FIGS. 5A-D, the GELFOAM® implants can be differentiated from surrounding tissue by its intense H and E (dark blue/purple) staining pattern. As compared to rhMIP-1α alone, the data suggests that MPV vCCI inhibits rhMIP-1α mediated recruitment of CD14⁺ cells, as indicated by a decrease in DAB positive (dark grey/brown) staining in and around the GELFOAM® implant (FIG. 5B). In an effort to quantify the levels of inhibition, the number of DAB positive pixels for each image was normalized to the PBS control and graphed as a migration index. FIG. 5E shows significant inhibition of rhMIP-1α mediated recruitment. These findings are consistent with the in vitro data and clearly indicated the MPV vCCI is a potent inhibitor of rhMIP-1α, both in vitro and in vivo.

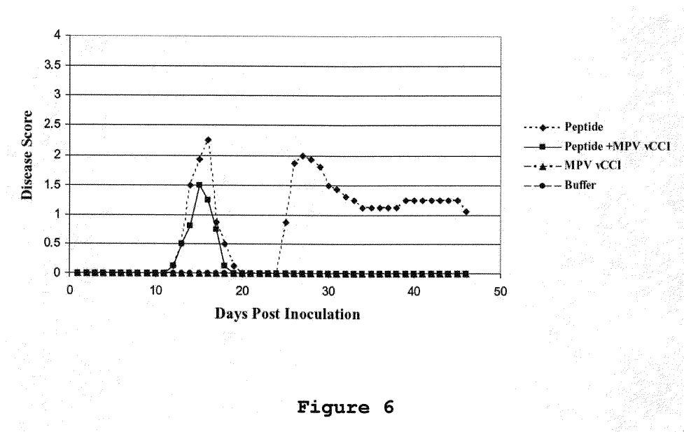

In order to assess the ability of MPV vCCI to treat a chemokine-mediated disease, the well described EAE mouse model was utilized. Four groups of mice were used: Group 1) Positive controls—mice that received PLP139-151 only; Group 2) Experimental group—mice that received recombinant MPV vCCI and PLP139-151; Group 3) MPV vCCI alone—mice receive MPV vCCI alone; and Group 4) Buffer alone—mice receive buffer alone. Groups 3 and 4 serve as negative controls. FIG. 6 shows that on day 12, both group 1 and 2 began to exhibit early signs of acute EAE and by day 16 the disease had peaked and both groups began to resolve the disease with complete recovery occurring by day 20. Interestingly, although administration of MPV vCCI did not stop or delay the onset of EAE, animals that received MPV vCCI showed a slight reduction in severity during the acute phase of disease. On day 24, animals of group 1 began to show signs of EAE relapse, lasting approximately 6 days. While the majority of animals fully recovered from EAE relapse, one animal developed chronic EAE, thus the consistent score from day 30 on. More importantly, none of the animals that received MPV vCCI showed any signs of relapse, which was confirmed in a second cohort of animals. Animals receiving MPV vCCI alone or buffer alone, showed no clinical signs of EAE or other pathologies. These data suggest that administration of recombinant MPV vCCI is capable of reducing, and possibly inhibiting, chemokine-mediated disease.

Chemokines play an important role in mediating the recruitment of leukocytes to sites of infection, and ultimately establishing effective innate and adaptive immune responses. As a result, many viruses encode proteins which subvert normal chemokine function. Herein, MPV vCCI was biologically characterized both in vitro and in vivo. MPV vCCI is shown herein to be expressed and secreted during MPV infection and that MPV vCCI efficiently inhibits rhMIP-1α mediated chemotaxis in both in vitro and in vivo assays. Furthermore, it is shown that MPV vCCI has the ability to halt relapsing EAE in mice, indicating that MPV vCCI is a novel therapeutic for the treatment of chemokine-mediated disease.

The MPV vCCI staining pattern seen during MPV infection is consistent with the cytoplasmic replication of poxviruses and represents the first time that any vCCI has been visualized using immunofluorescence. Furthermore, immunoprecipitation from infected supernatants and cell lysates clearly shows the presence of MPV vCCI. Interestingly, the presence of a single band in the lysate and a broadened band in the supernatant suggests that the secreted form of MPV vCCI may undergo some post-translational modification.

A commonality among all vCCI research from both the leporipoxvirus and orthopoxvirus genera is their ability to bind α- and β-chemokines. Structural analysis has determined that binding and subsequent inhibition is much stronger with β-chemokines. In fact, vCCI binding to α-chemokines occurs with such a low affinity that many question whether it is physiologically relevant (Smith et al. (1997) Virology, 236:316-27; Alcami et al. (1998) J. Immunol., 160:624-633). The work on MPV vCCI is consistent with these previous results, in that MPV vCCI forms a complex with rhMIP-1α resulting in a significant shift in MPV vCCI MWₐₚₚ. Although there is some debate as to the exact stoichiometry, stoichiometric analysis suggests that vCCI binds MCP-1 at nearly 1:1 ratio (Alcami et al. (1998) J. Immunol., 160:624-633; Seet et al. (2001) PNAS, 98:9008-9013). The data with rhMIP-1α provided herein is supported by these findings, in that at a 1:1 ratio, all of the MPV vCCI is migrating at the higher MWₐₚₚ, only when in excess does MPV vCCI migrate at the lower MWₐₚₚ.

Structural analysis of rabbitpox virus vCCI (RPV vCCI) complexed with human MIP-1β has provided significant insight as to a possible mechanism behind vCCI-mediated inhibition. Zhang et al. reported that RPV vCCI possess a number of important contacts with MIP-1β. In particular, the highly conserved vCCI resides Ser-182 to Thr-187 make “extensive contacts” with the chemokine (Zhang et al. (2006) PNAS, 103:13985-90). This region is critical for receptor binding, therefore high affinity association with vCCI appears to inhibit cc-chemokine receptor interaction. Along these lines, in vitro inhibition assays clearly demonstrate the inhibitory power of MPV vCCI as it completely blocks rhMIP-1α-mediated chemotaxis in a dose-dependent manner. This effect is dependent on MPV vCCI function, since heat inactivation of MPV vCCI restores rhMIP-1α-mediated migration to near rhMIP-1α alone levels. Although it is shown herein that MPV vCCI interacts with rhMIP-1α at approximately 1:1 stoichiometry (FIGS. 3B and 5), the in vitro inhibition assay requires 100 fold excess MPV vCCI for complete inhibition of rhMIP-1α mediated migration (FIG. 4). Without being bound by theory, this discrepancy is likely the result of using the human THP cells in the in vitro inhibition assay. Subtle differences between human and rhesus GPCRs may explain the requirement for excess vCCI to be present in order to achieve complete inhibition in THP cells. Regardless, MPV vCCI still exhibits inhibitory activity.

The in vivo studies on vCCI further confirm that the inhibitory potential is not limited to the in vitro setting. Although several reports have studied VV vCCI in mice and guinea pigs showing that vCCI can inhibit leukocyte recruitment in these animals, these are not natural host for VV, therefore slight differences may exist in the natural host (Smith et al. (1997) Virology, 236:316-27; Alcami et al. (1998) J. Immunol., 160:624-633; Reading et al. (2003) J. Immunol., 170:1435-42). Graham et al. investigated the inhibitory potential of RPV vCCI in rabbits showing a marked increase in leukocyte infiltrates when rabbits were infected with a vCCI knockout virus (Graham et al. (1997) Virology, 229:12-24). For the in vivo studies, two tests were performed, both of which utilized purified recombinant MPV vCCI. The first involved an in vivo inhibition assay in rhesus macaques using protein saturated GELFOAM® plugs. Although rhMIP1-α alone induces significant recruitment of CD14⁺ cells, when complexed with MPV vCCI, CD14⁺ recruitment was drastically reduced. Secondly, the ability of MPV vCCI to mitigate a chemokine-mediated disease was tested. Experimental allergic encephalomyelitis (EAE) is an induced disease in mice that closely mimics multiple sclerosis in human. Previous work by Karpus et al. has shown that administration of neutralizing antibodies for MIP-1α and MCP-1 causes a significant reduction in EAE disease, and therefore MIP-1α and MCP-1 must play an integral part in the establishment and progression of EAE (Karpus et al. (1995) J. Immunol., 155:5003-10). Prior to initiating the EAE study, it was confirmed that MPV vCCI interacts with several mouse chemokines, namely MIP-1α and MCP-1, via shift assay. These results were consistent with work by Smith et al., who showed that CPV vCCI bound with high affinity to mouse MCP-1, MCP-5, MIP-1α, MIP-1β, C10, and Eotaxin (Smith et al. (1997) Virology, 236:316-27). As the EAE study progressed into the acute phase, it was observed that mice receiving MPV vCCI exhibited reduced severity of disease compared to mice that did not receive MPV vCCI. More importantly, as mice from the positive control group began to exhibit signs of relapsing-remitting EAE (around day 24); the mice that received MPV vCCI did not and remained free of relapse until the end of the study.

This represents the first time that any vCCI has been shown to inhibit or mitigate a chemokine-mediated disease. As such, MPV vCCI is a therapeutic agent for the treatment of chemokine-mediated disease.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of inhibiting inflammation in a patient in need thereof, said method comprising administering a composition comprising a monkeypox virus viral CC-chemokine inhibitor (MPV vCCI) and at least one pharmaceutically acceptable carrier.

2. The method of claim 1, further comprising the administration of at least one additional anti-inflammatory agent.

3. A method for treating an inflammatory disorder in a patient in need thereof, said method comprising administering a composition comprising a monkeypox virus viral CC-chemokine inhibitor (MPV vCCI) and at least one pharmaceutically acceptable carrier.

4. The method of claim 3, further comprising the administration of at least one additional anti-inflammatory agent.

5. The method of claim 3, wherein said inflammatory disorder is an inflammatory lesion.

6. The method of claim 5, wherein said inflammatory lesion is associated with multiple sclerosis, organ transplant rejection, or tuberculosis.

7. The method of claim 3, wherein said inflammatory disease is encephalitis.

8. A composition for inhibiting inflammation, said composition comprising a monkeypox virus viral CC-chemokine inhibitor (MPV vCCI), at least one additional anti-inflammatory agent, and at least one pharmaceutically acceptable carrier.

9. The method of claim 1, wherein the amino acid sequence of said MPV vCCI has at least 95% identity with SEQ ID NO: 7.

10. The method of claim 3, wherein the amino acid sequence of said MPV vCCI has at least 95% identity with SEQ ID NO: 7.

11. The composition of claim 8, wherein the amino acid sequence of said MPV vCCI has at least 95% identity with SEQ ID NO: 7.


SEQUENCE LISTING (11 sequences)

SEQ ID NO: 1 - DNA, 26 bp, Artificial Sequence
  Primer
catatgatccctaccagtcttcagca

SEQ ID NO: 2 - DNA, 51 bp, Artificial Sequence
  Primer
ctcgagtcatcagtggtggtggtggtggtggacacatgctttgagttttgt

SEQ ID NO: 3 - DNA, 22 bp, Artificial Sequence
  Primer
aacaaacatcacatgggaatcg

SEQ ID NO: 4 - DNA, 22 bp, Artificial Sequence
  Primer
cgattcccatgtgatgtttgtt

SEQ ID NO: 5 - DNA, 23 bp, Artificial Sequence
  Primer
catatggctgacaccccgacctc

SEQ ID NO: 6 - DNA, 51 bp, Artificial Sequence
  Primer
ctcgagtcatcagtggtggtggtggtggtgcacggcactcagctctaggtc

SEQ ID NO: 7 - protein, 246 aa, Monkeypox virus
MKQYIVLACMCLVAAAMPTSLQQSSSSCTEEENKHHMGIDVIIKVTKQDQTPTNDKICQSVTEVTETEDDEVSEEVVKGDPTTYYNIVGAGLNMNFGFTKCPKISSISESSDGNTVNTRLSSVSPGQGKDSPAITREEALAMIKDCEMSIDIRCSEEEKDSDIKTHPVLGSNISHKKVSYKDIIGSTIVDTKCVKNLEFSVRIGDMCEESSELEVKDGFKYVDGSASEGATDDTSLIDSTKLKACV

SEQ ID NO: 8 - protein, 253 aa, Variola virus
MKQYIVLACMCLAAAAMPASLQQSSSSCTEEENKHYMGIDVIIKVTKQDQTPTNDKICQSVTEITESESDPEVESEDDSTSVEDVDPPTTYYSIIGGGLRMNFGFTKCPQIKSISESANGNAVNARLSSVPPGQGKDSPAITRVEALAMIKDCELSIDIRCSEEEKDSDIQTHPVLESNISHKKVSYEDIIGSTIVDTKCVKNLEFSVRIGDMCKESSDLEVKDGFKYVDGSVSEGVTDDTSLIDSTKLKSCV

SEQ ID NO: 9 - protein, 246 aa, Cowpox virus
MKQIVLACICLAAVAIPTSLQQSFSSSSSCTEEENKHHMGIDVIIKVTKQDQTPTNDKICQSVTEVTESEDESEEVVKGDPTTYYTVVGGGLTMDFGFTKCPKISSISEYSDGNTVNARLSSVSPGQGKDSPAITREEALSMIKDCEMSINIKCSEEEKDSNIKTHPVLGSNISHKKVSYEDIIGSTIVDTKCVKNLEISVRIGDMCKESSELEVKDGFKYVDGSASEDAADDTSLINSAKLIACV

SEQ ID NO: 10 - protein, 258 aa, Rabbitpox virus
MKQYIVLACMCLAAAAMPASLQQSSSSSSSCTEEENKHHMGIDVIIKVTKQDQTPTNDKICQSVTEITESESDPDPEVESEDDSTSVEDVDPPTTYYSIIGGGLRMNFGFTKCPQIKSISESADGNTVNARLSSVSPGQGKDSPAITHEEALAMIKDCEVSIDIRCSEEEKDSDIKTHPVLGSNISHKKVSYEDIIGSTIVDTKCVKNLEFSVRIGDMCKESSELEVKDGFKYVDGSASKGATDDTSLIDSTKLKACV

SEQ ID NO: 11 - protein, 244 aa, Vaccinia virus, Copenhagen strain
MHVPASLQQSSSSSSSCTEEENKHHMGIDVIIKVTKQDQTPTNDKICQSVTEITESESDPDPEVESEDDSTSVEDVDPPTTYYSIIGGGLRMNFGFTKCPQIKSISESADGNTVNARLSSVSPGQGKDSPAITREEALAMIKDCEVSIDIRCSEEEKDSDIKTHPVLGSNISHKKVSYEDIIGSTIVDTKCVKNLEFSVRIGDMCKESSELEVKDGFKYVDGSASEGATDDTSLIDSTKLKACV